(19) United States
(12) Patent Application Publication
(10) Pub. No.: US 2021/0330711 A1
(43) Pub. Date: Oct. 28, 2021

(54) COMPOSITIONS WITH ANTIVIRAL ACTIVITY AND RELATED METHODS

(71) Applicant: FETTECH, LLC, Davie, FL (US)

(72) Inventor: Clay FETTE, Davie, FL (US)

(73) Assignee: FETTECH, LLC, Davie, FL (US)

(21) Appl. No.: 17/237,239

(22) Filed: Apr. 22, 2021

Related U.S. Application Data
(60) Provisional application No. 63/013,789, filed on Apr. 22, 2020.

(51) Int. Cl. A61K 35/26; A61K 35/42

(52) U.S. Cl. CPC A61K 35/26; A61K 35/42

(57) ABSTRACT

Compositions that include extracellular matrix (ECM) materials having antiviral activity and methods of use thereof are disclosed. The compositions may be administered to a subject in need thereof, e.g., subject having a viral infection or being suspected of having a viral infection. The virus may be an enveloped virus, such as a coronavirus.

### CROSS-REFERENCE TO RELATED APPLICATIONS

This application claims priority to U.S. Provisional Application No. 63/013,789, filed on Apr. 22, 2020, which is incorporated by reference herein in its entirety.

### TECHNICAL FIELD

The present disclosure generally relates to compositions comprising biomaterials with antiviral activity and methods of use thereof. The compositions herein comprise one or more extracellular matrix (ECM) materials.

### BACKGROUND

Viruses are a serious public health concern, particularly given the highly-infectious nature of many viruses. The global COVID-19 pandemic, caused by the spread of severe acute respiratory syndrome coronavirus 2 (SARS-CoV-2), emphasizes the threat posed by viral infections. A virus is a chain of nucleic acids (DNA or RNA) which lives in a host cell, uses parts of the cellular machinery to reproduce, and releases the replicated nucleic acid chains to infect more cells. Viruses may be enveloped or non-enveloped, where the envelope refers to an outermost layer of phospholipids and proteins of the virus, including glycoproteins that bind to receptor sites of the host cell. More than 200 viruses are known to cause disease in humans.

### SUMMARY

The present disclosure includes compositions and related methods of treatment, including use of the compositions for treating viral infections. For example, the present disclosure includes a method of treating a subject, the method comprising administrating a composition to the subject, wherein the composition comprises at least one extracellular matrix (ECM) material, the at least one ECM material being derived from tissue chosen from spleen, lung, gall bladder, bone marrow, pancreas, liver, or a mixture thereof, the at least one ECM material retaining native bioactive components of the tissue. The subject may have infected or suspected of being infected with a virus. For example, the subject may have an infectious disease. The composition may reduce a viral load of the subject. For example, the composition may inactivate at least a portion of a virus population of the subject. According to some aspects of the present disclosure, the virus is an enveloped virus. For example, the virus may be a coronavirus or an adenovirus, such as, e.g., severe acute respiratory syndrome-associated coronavirus (SARS-CoV), severe acute respiratory syndrome-associated coronavirus 2 (SARS-CoV-2), or Middle East respiratory syndrome coronavirus (MERS-CoV). The subject may be a human or a non-human mammal. The composition administered to the subject may comprise at least two different types of ECM materials. For example, the composition may comprise spleen ECM material and lung ECM material. According to some aspects of the present disclosure, the composition may be in particulate form, gel form, or liquid form. With respect to administration, the composition may administered via inhalation or intravenously, for example.

The present disclosure also includes a method of treating a subject, comprising administrating a composition to the subject, wherein the composition comprises at least two different extracellular matrix (ECM) materials, the ECM materials being derived from a tissue independently chosen from spleen tissue, lung tissue, gall bladder tissue, bone marrow tissue, pancreas tissue, or liver tissue; wherein the subject is infected or suspected of being infected with a virus, and wherein the virus is an enveloped virus. The virus may be a coronavirus or an adenovirus, for example. In some examples, the subject may have an infectious viral disease. The composition may be administered via inhalation or intravenously.

The present disclosure also includes a method of treating a subject, comprising administrating a composition to the subject, wherein the composition comprises at least one extracellular matrix (ECM) material, the at least one ECM material being derived from a tissue chosen from spleen tissue, lung tissue, gall bladder tissue, bone marrow tissue, pancreas tissue, or liver tissue; wherein the subject is infected or suspected of being infected with a virus; and wherein the subject is a human or a non-human mammal. The virus may be an enveloped virus such as, e.g., coronavirus or an adenovirus.

### DETAILED DESCRIPTION

The present disclosure includes biomaterials with antiviral activity and methods of use thereof. For example, the present disclosure includes methods of treating a subject, e.g., by administering the biomaterial(s) to the subject. The compositions and methods herein may be useful to reduce viral load and/or treat health conditions associated with viruses, e.g., infectious diseases such as viral infections, including respiratory infections.

The terms “approximately” and “about” refer to being nearly the same as a referenced number or value. As used herein, the terms “approximately” and “about” generally should be understood to encompass ±10% of a specified amount or value.

As used herein, the term “therapeutically-effective amount” relates to an amount of a substance (e.g., an agent, compound, material, etc.) that leads to the desired therapeutic effect(s), and the term “pharmaceutically-effective amount” relates to an amount of a substance (e.g., an agent compound, material, etc.) that leads to the desired pharmacological effect(s). While individual patient needs may vary, determination of optimal ranges for effective amounts of the substances described herein (e.g., ECM materials, growth factors, structural proteins, therapeutic agents, pharmaceutical agents, antimicrobial agents, etc.) is within the skill of the art. For example, suitable amounts or dosages of the substances herein may be selected in accordance with a variety of factors, including the type, age, weight, sex, diet, medical condition, and/or medical history of the patient.

The extracellular matrix (ECM) is a complex structural material found within tissues that surround and support cells. The ECM is generally made up of three major classes of biomolecules: structural proteins such as collagen and elastin; other proteins such as laminin, fibronectin, and various growth factors; and proteoglycans. ECM components that may have therapeutic efficacy include, but are not limited to, interleukins, growth factors, exosomes, protein fragments, and binding site motifs. The compositions herein may comprise and/or may be derived from ECM materials such that the compositions retain native ECM components that have therapeutic efficacy, and in particular, antiviral activity. Thus, for example, the compositions herein may be useful in treating a viral infection. The subjects treated herein may be a human or a non-human mammal.

For example, a method of the present disclosure includes administering a composition comprising one or more ECM materials to a subject who has a virus or viral infection, or to a subject who is suspected of having a virus and/or viral infection. For example, a human or non-human mammal who has come into close contact with another human or non-human mammal known to have a viral infection may be suspected of being exposed to the virus and developing a viral infection. Such subjects also may be treated by with the compositions and methods herein.

The compositions herein may comprise ECM materials and/or components thereof, wherein the ECM materials and/or components thereof have antiviral activity. The ECM materials may contain naturally occurring components embedded on and within the structure of the various ECM components. It may be advantageous to extract or isolate desired components and/or structures that are present in and on the native ECM. Examples of such components may include, but are not limited to, interleukins, growth factors, exosomes, protein fragments, and binding site motifs. Some of the components may be separated from (e.g., extracted from) a native ECM material and formulated into an extract or isolate of the original native ECM material. These extracts or isolates may be useful for antiviral activity and/or other therapeutic activity. For example, the compositions herein may comprise isolates and/or distillates ECM materials.

As discussed further below, the composition may comprise at least one, including two or more, different types of ECM materials and/or may be formulated for administration to a subject in one or more suitable forms such as, e.g., particulate, solution, or gel form.

The compositions and biomaterials thereof disclosed herein may have antiviral activity against various types of viruses including, but not limited to, viruses associated with infection, disease, and/or other health conditions affecting humans and non-human mammals. The subject treated with the compositions herein may have a viral infection or may be suspected of having or developing a viral infection (e.g., having been exposed to another subject known or suspected of having a viral infection). The viral infection may be associated with an infectious disease. Such infectious diseases include, but are not limited to, Ebola, COVID-19, and influenza.

According to some examples herein, the virus affected by the compositions and methods herein may be an enveloped virus. Without being bound by theory, it is believed that the ECM material(s) and/or components thereof may bind or interfere with the envelope of the virus. Thus, for example, the compositions and methods herein may be used to treat a subject infected by, or otherwise carrying a viral load of, an enveloped virus. Administering the compositions herein may reduce a viral load of the subject. For example, the composition may reduce the viral load by at least 10%, at least 15%, at least 20%, at least 25%, or at least 30%. The reduction in viral load may be measured by comparing the initial viral load (copies per mL) prior to administration of the composition to the viral load at a given time period from the date of administration. The given time period may be 24 hours, 1 week, 2 weeks, or 1 month, for example.

Exemplary viruses that may be affected by the compositions and methods herein include, but are not limited to, viruses of the Coronaviridae family, the Adenoviridae family, the Parvoviridae family, the Picornaviridae, family and/or the Retroviridae family. Exemplary viruses include, for example, coronaviruses, e.g., human coronaviruses such as severe acute respiratory syndrome-associated coronavirus (SARS-CoV), SARS-CoV-2, and Middle East respiratory syndrome coronavirus (MERS-CoV). Other exemplary viruses include, for example, encephalomyocarditis virus (EMC), porcine parvovirus (PPV), pseudorabies virus (PrV), and xenotropic murine leukemia virus (X-MuLV). EMC is a 28-30 nm, non-enveloped, RNA-containing virus of the Picornaviridae family and has an icosahedral shape. PPV is an 18-28 nm, non-enveloped, DNA-containing virus of the Parvoviridae family and has an icosahedral shape. PrV is a 150-200 nm, enveloped, DNA-containing virus of the Adenoviridae family and has a spherical shape. X-MuLV is an 80-130 nm, enveloped, RNA-containing virus of the Retroviridae family and has a spherical shape.

According to some aspects of the present disclosure, the subject to be treated by the compositions and methods herein may have a coronavirus, e.g., an infection caused by a coronavirus, or the subject may be suspected of having a coronavirus, e.g., an infection caused by a coronavirus. The coronavirus may be, for example, SARS-CoV, SARS-CoV-2, or MERS-CoV. In some examples, the subject has or is suspected of having COVID-19.

Exemplary routes of administration of such materials include, for example, inhalation and intravenous (IV) injection. The materials and/or components of the materials may come in direct contact with the virus, e.g., in a subject's respiratory system or bloodstream. For example, the subject may have one or more viruses present in the bloodstream, respiratory system (including the lungs), and/or mucous membranes. The composition may be administered in a variety of forms. For example, the composition may be in particulate, liquid (e.g., as a solution), and/or gel form, e.g., depending on the desired route of administration.

In some examples, the composition may be in particulate form. The composition in particulate form optionally may be administered via inhalation. For example, when the composition administered is in a powder or particulate form, the particulates may be micronized to an appropriate particle size for inhalation. In an exemplary process of producing a particulate composition comprising ECM material, a suitable technique may be used to avoid adverse effects (e.g., loss of bioactivity) on the active component(s) in the materials and/or generating too much heat which may damage the material. Prior to forming particulates or powder the ECM material(s) may be lyophilized.

In some embodiments, the composition may be in liquid (e.g., as a solution) form, while in other embodiments the composition may be in gel form. The composition in liquid or gel form optionally may be administered intravenously. For example, when the ECM materials are formulated in a gel form, the gel may optionally be diluted and formulated to provide a suitable viscosity and biocompatibility to be IV injected or added to an IV drip. Gels may be prepared and optionally frozen so that the lyophilized ECM powder may be produced. Powders prepared via gel may facilitate formulation into an inhalable particulate. Alternatively, the liquid or gel may be kept and administered to the subject, e.g., introduced into the patient's respiratory system, via nasal spray, nebulizer, etc., which may be able to use suspended particulate.

As mentioned above, suitable dosages may be selected in accordance with a variety of factors, including the type, age, weight, sex, diet, medical condition, and/or medical history of the patient. An exemplary dose according to some aspects of the present disclosure is about 1 mg to about 20 mg, such as about 5 mg to about 15 mg or about 7 mg to about 12 mg, e.g., about 10 mg, administered at least once a day. For example, the composition may be administered 1-10 times per day, with 3-10 days of treatment. The days of treatment may be consecutive or may be at periodic intervals of 1, 2, or 3 days between treatments. The composition may be administered by inhalation or intravenously. In some examples, the composition is administered by inhalation in a dose of about 5 mg to about 15 mg once per day for 3-10 consecutive days. In some examples, the composition is administered by inhalation in a dose of about 7 mg to about 12 mg twice per day for 3-10 consecutive days. In some examples, the composition is administered by inhalation in a dose of about 10 mg from 1-5 times per day for 3-10 days with 1 or 2 days between each treatment.

ECM materials suitable for the present disclosure may be derived from any mammalian source tissue comprising ECM or other collagen-based materials, including, but not limited to, tissues of the spleen, kidney, liver, lung, pancreas, gall bladder, stomach, pericardium, lymph node, bone marrow, dermis, placenta, amniotic sac, dura mater, and any combinations thereof. ECM source material may be used in whole or in part. Examples of types of native tissues suitable for the present disclosure include, but are not limited to, porcine, bovine, ovine, and human tissue. The ECM materials may be derived from tissue that is allogeneic, autologous, or xenogeneic to the subject (e.g., patient) being treated. The present disclosure may include any of the compositions and/or features or uses thereof disclosed in U.S. Pat. No. 9,238,090, which is incorporated by reference herein in its entirety.

According to some aspects of the present disclosure, the composition may comprise ECM materials derived from two or more different tissue sources and/or two or more different native tissues. For example, the tissue sources can be from the same species (e.g., ECM materials derived from different types of tissues of the same mammal), from different species (e.g., ECM materials derived from the same type of tissue of different types of mammals), or both (e.g., ECM materials derived from different types of tissues of different types of mammals). In some embodiments, the composition may comprise spleen ECM and/or lung ECM from the same species or different species. For example, embodiments of the present disclosure may include, but are not limited to, compositions comprising the following: Porcine spleen ECM and porcine lung ECM; Bovine spleen ECM and porcine spleen ECM; Bovine lung ECM and porcine spleen ECM; Bovine lung ECM, bovine spleen ECM, and porcine spleen ECM.

The composition may comprise spleen ECM and at least one other ECM material chosen from lung, gall bladder, bone marrow, pancreas, or liver. For example, the composition may comprise spleen ECM, lung ECM, and/or at least one other ECM material. Other combinations of ECM materials will be apparent in view of the disclosure herein. In some embodiments, the composition may comprise a plurality of ECM materials, wherein at least one of the ECM materials comprises a different variety of components and/or a different amount or concentration of a given component than another ECM material in the composition. Further, in some embodiments, the composition may comprise two or more ECM materials that comprise some or all of the same variety of components, but different amounts or concentrations of those components. Combining two or more types of ECM materials may provide compositions uniquely tailored to the specific needs of a subject (e.g., patient). The compositions may be designed to take advantage of the signaling components naturally occurring in the tissues from which the ECM materials are derived, including, but not limited to, vascular endothelial growth factor (VEGF), fibroblast growth factor (FGF), epidermal growth factor (EGF), transforming growth factors beta (TGF-β), platelet-derived growth factor (PDGF), and/or insulin-like growth factor (IGF). For example, the composition may comprise two or more ECM materials in particulate form, with the ratios of the ECM materials adjusted to provide a composition with a desired content of desired bioactive components, including, but not limited to, growth factor and/or other signaling components. The composition may be administered in particulate form, or may be administered (e.g., injected) as a liquid, gel, suspension, or particulates in combination with a carrier (e.g., liquid solution).

In some examples, the composition may comprise two or more ECM materials that comprise some or all of the same variety of components and/or substantially the same amount or concentration (or a similar amount or concentration) of a given component as another ECM material in the composition. These components may include proteins, glycoproteins, glycosaminoglycans, proteoglycans, cytokines, and/or growth factors. For example, the composition may comprise ECM materials having substantially the same, similar, or different amounts of one or more of the following components: collagen, elastin, fibronectin, laminin, heparin sulfate, fibroblast growth factor (FGF), vascular endothelial growth factor (VEGF), transforming growth factors alpha (TGF-α), transforming growth factors beta (TGF-β), platelet-derived growth factor (PDGF), epidermal growth factor (EGF), keratinocyte growth factor (KGF), bone morphogenetic proteins (BMPs), epidermal growth factor (EGF), brain-derived neurotrophic factor (BDNF), growth differentiation factor-9 (GDF9), hepatocyte growth factor (HGF), insulin-like growth factor (IGF), nerve growth factor (NGF), skeletal growth factor (SGF), osteoblast-derived growth factor (BDGF), cytokine growth factors (CGF), stem cell derived factor (SDF), stem cell factor (SCF), placental growth factor (PGF), and/or interleukins of any type and within any family (e.g., IL-1, IL-2, etc.).

Compositions according to the present disclosure may comprise the same or different amounts of each ECM material. In some embodiments, the composition may comprise two different ECM materials, e.g., in a weight ratio ranging from about 50:50 (i.e., 1:1) to about 5:95 (i.e., 1:19). For example, the composition may comprise a 50:50 weight ratio of two different ECM materials (e.g., spleen ECM and lung ECM), or a weight ratio of 60:40, 70:30, 80:20, 90:10, 40:60, 30:70, 20:80, 10:90, or any other weight ratios in between. For example, the composition may comprise lung and spleen tissues in a 1:3 weight ratio (e.g., about 25% wt. lung ECM and about 75% wt. spleen ECM, or about 25% wt. spleen ECM and about 75% wt. lung ECM). In some embodiments, the composition may comprise more than two different ECM materials, e.g., three, four, five or more ECM materials, in equal or unequal amounts. For example, the composition may comprise three different ECM materials having a weight ratio of 40:40:20, 30:30:40, or 20:20:60, among other possible weight ratios. In some embodiments, for example, the composition may comprise about 25% wt. spleen ECM, about 25% wt. lung ECM, and about 50% wt. gall bladder ECM. Any desired weight ratio may be selected based on the desired final composition.

As mentioned above, the compositions herein may be in particulate form. For particulate compositions (also referred to as powders herein), the particles may range from about 100 nm or less to about 2000 μm or more in diameter. The particle size distribution of a composition may be selected based on the desired application. For example, particles ranging from about 100 nm to about 5 μm in diameter, e.g., from about 1 μm to about 5 μm in diameter, may be suitable for administration via inhalation. Further, for example, particles ranging from about 50 μm to about 100 μm in diameter may be suitable for a variety of therapeutic injection applications. These size ranges are intended as general guidelines only, and may vary according to the medical application and/or particular needs of a patient. Particle size may be measured by laser diffraction, for example.

The composition may comprise ECM materials in particulate form, e.g., for administration via inhalation. The particles may range from about 100 nm to about 5 μm in diameter (e.g., an average diameter for a distribution of particles), such as from about 1 μm to about 5 μm in diameter, or from about 100 nm to about 2.5 μm in diameter. In some embodiments, for example, the composition may comprise particles having substantially the same size, e.g., a particle diameter of about 100 nm, about 300 nm, about 500 nm, about 1 μm, about 2.5 μm, or about 5 μm. In some embodiments, the composition may comprise particles having a size distribution with an average or median diameter ranging from about 300 nm to about 5 μm, e.g., about 300 nm, about 500 nm, about 1 μm, about 2.5 μm, or about 5 μm. The size distribution may be unimodal or bimodal. Compositions according to the present disclosure may be prepared by mixing different ECM materials together in particulate form. ECM particulates may be prepared by chopping, cutting, pulverizing, milling, or grinding the ECM material with a suitable device such as a blender, a hammer mill, a knife mill, a centrifugal mill, or a roller crusher, for example, to form particles.

The composition may comprise two or more ECM materials in particulate form. For example, the composition may comprise a first set of particles of a first ECM material, and a second set of particles of a second ECM material, wherein the first and second sets of particles may have the same or different diameters or size distributions. According to some aspects, the composition may comprise particles of lung and spleen ECM ranging from about 1 μm to about 5 μm in diameter for delivery to the lungs of a patient via inhalation.

Particles may be used in the composition directly, or may be combined with a suitable liquid or gel to form a paste or dispersion for administration. For example, one or more ECM materials may be dissolved into a liquid or gel, e.g., via enzymatic digestion or other biological or chemical process. The liquid or gel may be used in the composition directly, or may be combined with a suitable liquid or gel to form an emulsion for administration. In some embodiments, the structure of the ECM material(s) may be modified, e.g., via a chemical process to denature proteins of the ECM, to provide for increased porosity.

The composition may be formulated for administration via injection, e.g., in liquid or gel form. ECM particulates, gels, and/or particulate and gel combinations may be used as an injectable for a variety of therapeutic applications, including treatment of viral infections. Gels of the materials herein may be produced by digesting (e.g., via digestive enzymes) or chemically processing ECM materials or components thereof into gel form, which may be combined with ECM particles or other additional ECM materials or components thereof. According to some aspects of the present disclosure, the composition may comprise a weight ratio of particulate lung ECM and particulate spleen ECM, e.g., a 1:2 weight ratio, 1:1 weight ratio, or 2:1 weight ratio, among other examples, suspended within an ECM gel as a carrier (e.g., a lung ECM gel, spleen ECM gel, or lung/spleen mixture ECM gel).

The particulate composition may be used to treat the respiratory system, to treat a respiratory disorder, and/or to deliver a pharmaceutical agent such as a steroid, an antifungal agent, an antimicrobial agent, and/or a bronchodilator, for uptake via the respiratory system. In some embodiments, for example, the composition may be used to treat asthma, lung infections including viral infections, and/or a lung injury, such as damage to the lungs from inhalation of damaging gases, smoke, or chemical or biological agents. In some embodiments, the composition may be administered to mitigate the effects of diseases that cause damage to the lungs, e.g., as part of a dosing regimen. In some examples, the composition may serve as a delivery vehicle for a pharmaceutical or therapeutic agent. For example, the pharmaceutical or therapeutic agent may be incorporated into the ECM material during processing such that the agent is associated with the particles, such as a coating.

The following examples are intended to illustrate the present disclosure without, however, being limiting in nature. It is understood that the present disclosure encompasses additional embodiments consistent with the foregoing description and following examples.

### EXAMPLES

### Example 1

A particulate material according to the present disclosure was tested against different viruses to assess properties of the composition. In these studies, the test material was a micronized powder of a combination of porcine spleen and lung ECM material (a 1:1 weight ratio comprising 50% wt. porcine spleen ECM material and 50% wt. porcine lung ECM material) with a particle size distribution of approximately 1-1000 μm. The test material was exposed to each virus of a select panel of four viruses representing a range of viral characteristics, as follows:

1) Encephalomyocarditis virus (EMC, EMC strain). EMC can serve as a model for foot- and mouth disease virus (FMDV) and as a model for hepatitis E virus.

2) Porcine parvovirus (PPV, NADL-2 strain). PPV can serve as a model for other parvoviruses (Parvoviridae family) such as the bovine parvovirus.

3) Pseudorabies virus (PrV, SHOPE strain). PrV can serve as a model for other herpesviruses such as cytomegalovirus (CMV), herpes simplex virus type 1 (HSV-1), Epstein-Barr virus (EBV), and Varicella-Zoster virus (VZV).

4) Xenotropic murine leukemia virus (X-MuLV, pNFS Th1 strain). X-MuLV can serve as a model for retroviruses such as porcine endogenous retrovirus (PERV).

As summarized in Table 1 below, two of the viruses are enveloped viruses (PrV and X-MuLV), and two of the viruses are non-enveloped viruses (EMC and PPV). Additionally, two of the viruses are RNA-containing (EMC and X-MuLV), and two of the viruses are DNA-containing (PPV and PrV).

TABLE 1

| | | | | Approx. | |
|---|---|---|---|---|---|
| | | Enve- | | Size | |
| Virus | Virus Family | lope | Geno | (nm) | Shape |
| EMC | Picornaviridae | No | RNA | 28-30 | Icosahedral |
| PPV | Parvoviridae | No | DNA | 18-26 | Icosahedral |
| PrV | Adenoviridae | Yes | DNA | 150-200 | Spherical |
| X-MuLV | Retroviridae | Yes | RNA | 80-130 | Spherical |

Portions of the test material were exposed to stock solutions of the viruses. The stock solution for each virus first was sonicated and filtered. For each virus, at least 1 gram of the test material was spiked with the appropriate stock virus solution at a ratio of 1 mL virus/1 gram of material. The samples were incubated at 2-8° C. for 15 minutes to allow for absorption of the virus. Following virus incubation, the samples were resuspended in 9 mL of EMEM/1 gram of sample and mixed thoroughly (EMEM=Eagle's minimum essential medium, cell culture medium). The solution was removed and saved. The sample material was mixed in a small volume of the solution. The pooled solution was confirmed to be pH 6.5-7.5 and filtered (0.45 μm). Infectivity studies (cell-based toxicity studies and cell-based interference studies) were done immediately on portions of each sample. The remaining sample was divided into multiple aliquots, snap frozen and stored as back-ups at or below −60° C.

Cell-based toxicity studies were conducted to assess toxicity to Vero indicator cells (EMC-incubated sample), ST indicator cells (PPV-incubated sample), CV-1 indicator cells (PrV-incubated sample), and PG4 indicator cells (X-MuLV sample). For each cell line, all of the samples were tested in duplicate for cytotoxicity at full strength and at 3-fold, 5-fold, 10-fold, 30-fold, 50-fold, 100-fold, 300-fold, 500-fold and 1000-fold dilutions via the standard toxicity procedure for each virus. For all indicator cell lines, samples that reduced the cell numbers to less than 80% of the controls were considered cytotoxic. All samples were found to be not toxic.

Cell-based interference studies were conducted to assess possible interference on the assay systems used to titrate the viruses. Multiple dilutions of each sample were prepared with serum-free medium. Each dilution of the samples was spiked with 10% v/v of one of three concentrations of the appropriate virus stock solution. Aliquots of serum-free medium were be spiked with 10% v/v of each of the three concentrations of the appropriate virus stock solution as controls. Each of the generated spiked samples was assayed in triplicate. Samples which altered the virus titers by >0.5 log₁₀ were considered to interfere. No interference was found in any of the samples.

To assess virus inactivation by the test material, five samples for each type of virus were prepared:
1) T₀: Sample prepared as described above on the day of virus inactivation testing.
2) E-Beam Treated Sample: Samples of the test material were spiked with each stock virus solution at a ratio of 1 mL virus/1 gram of material and incubated at 2-8° C. for 15 minutes as described above. The spiked test material was then packaged and sealed in a shipping container and shipped at ambient temperature to a sterilization facility. Upon arrival at the sterilization facility, the shipping container was irradiated with gamma radiation (˜25 KGy). The container was then shipped to the testing facility for virus inactivation testing. Upon arrival at the testing facility, the sample was further processed as described above (i.e., resuspended in 9 mL of EMEM/1 gram of sample; mixed thoroughly; removing the solution; mixing the sample material in a small volume of solution; confirming pH and filtering). A portion of the sample was tested for viral inactivation immediately. The remaining sample was divided into multiple aliquots, snap frozen and stored as back-ups at or below −60° C.
3) E-Beam Untreated Sample: Samples of the test material were prepared as described above for the “E-Beam Treated Sample” but not irradiated at the sterilization facility. A portion of each sample was tested for viral inactivation immediately after processing at the testing facility. The remaining sample was divided into multiple aliquots, snap frozen and stored as back-ups at or below −60° C.
4) Shipping Control: For each sample, a 9 ml aliquot of EMEM was spiked with 1 ml of the appropriate stock virus solution. These samples did not contain the test material. Each sample was shipped at ambient temperature to the sterilization facility but was not irradiated, and then shipped at ambient temperature to the testing facility. Upon arrival at the testing facility, each sample was confirmed to be pH 6.5-7.5 and filtered as described above. A portion of each sample was tested immediately. The remaining material of each sample was divided into multiple aliquots, snap frozen, and stored at or below −60° C. Shipments were sent overnight and scheduled during the week to avoid weekend shipping delays, making the estimated shipment time to the sterilization facility and then to the testing facility approximately 4-5 days total.

The stock virus solutions served as positive controls, wherein an aliquot of each sonicated and filtered stock virus solution was assayed. Cell culture medium used for each virus titration served as a negative control.

To measure the amount of virus present in each sample (i.e., To, E-Beam Treated, E-Beam Untreated, Shipping Control, and positive and negative controls) aliquots were diluted in serum-free medium (10°, 10⁻¹, 10⁻², 10⁻³, 10⁻⁴, 10⁻⁵, 10⁻⁶, 10⁻⁷, and 10⁻⁸) and assayed as follows.
EMC: Each dilution of EMC-containing samples was assayed in multiple wells for infectious viral particles by EMC plaque assay using Vero indicator cells.
PPV: Each dilution of PPV-containing samples was assayed in multiple wells for infectious viral particles by PPV plaque assay using ST indicator cells.
PrV: Each dilution of PrV-containing samples was assayed in multiple wells for infectious viral particles by PrV plaque assay using CV-1 indicator cells.
X-MuLV: Each dilution of X-MuLV-containing samples was assayed in multiple wells for infectious viral particles by X-MuLV plaque assay using PG4 indicator cells.

The positive controls confirmed the presence of virus and the negative controls the absence of virus. Results for the To, E-Beam Treated, E-Beam Untreated, and Shipping Control samples are summarized below, wherein Table 2 reports results for the EMC-incubated samples, Table 3 reports results for the PPV-incubated samples, Table 4 reports results for the PrV-incubated samples, and Table 5 reports results for the X-MuLV-incubated samples. The values are reported with the 95% confidence levels, as applicable.

TABLE 2
EMC

| | | | Log₁₀ | |
|---|---|---|---|---|
| | Mean | | Adjusted Titer | Log₁₀ |
| Sample | PFU | Dilution | (PFU) | Reduction |
| Stock Virus | 40.33 ± 6.25 | 1.0E+7 | 8.91 ± 0.06 | N/A |
| Control | | | | |
| T₀ | 29.67 ± 1.43 | 1.0E+6 | 8.77 ± 0.02 | 0.14 ± 0.06 |
| E-Beam | 0.00 | 1.0E+0 | <1.30 | >7.47 ± 0.02 |
| Treated | | | | |
| E-Beam | 29.33 ± 7.99 | 1.0E+4 | 6.77 ± 0.10 | 2.00 ± 0.10 |
| Untreated | | | | |
| Shipping | 24.00 ± 6.57 | 1.0E+6 | 8.68 ± 0.11 | 0.23 ± 0.13 |
| Control | | | | |

TABLE 3
PPV

| | | | Log₁₀ | |
|---|---|---|---|---|
| | Mean | | Adjusted Titer | Log₁₀ |
| Sample | PFU | Dilution | (PFU) | Reduction |
| Stock Virus | 39.33 ± 15.78 | 1.0E+6 | 7.90 ± 0.14 | N/A |
| Control | | | | |
| T₀ | 32.67 ± 15.18 | 1.0E+5 | 8.29 ± 0.17 | None |
| E-Beam | 27.67 ± 7.59 | 1.0E+1 | 4.22 ± 0.11 | 4.07 ± 0.20 |
| Treated | | | | |
| E-Beam | 51.00 ± 4.97 | 1.0E+4 | 7.49 ± 0.04 | 0.80 ± 0.17 |
| Untreated | | | | |
| Shipping | 32.33 ± 7.17 | 1.0E+5 | 7.81 ± 0.09 | 0.09 ± 0.17 |
| Control | | | | |

TABLE 4
PrV

| | | | Log₁₀ | |
|---|---|---|---|---|
| | Mean | | Adjusted Titer | Log₁₀ |
| Sample | PFU | Dilution | (PFU) | Reduction |
| Stock Virus | 65.33 ± 7.99 | 1.0E+6 | 8.12 ± 0.05 | N/A |
| Control | | | | |
| T₀ | 19.33 ± 6.25 | 1.0E+5 | 7.59 ± 0.12 | 0.53 ± 0.13 |
| E-Beam | 0.00 | 1.0E+0 | <1.30 | >6.29 ± 0.12 |
| Treated | | | | |
| E-Beam | 1.00 ± 0.00 | 1.0E+0 | 1.30 | 6.29 ± 0.12 |
| Untreated | | | | |
| Shipping | 23.33 ± 5.17 | 1.0E+5 | 7.67 ± 0.09 | 0.45 ± 0.10 |
| Control | | | | |

TABLE 5
X-MuLV

| | | | Log₁₀ | |
|---|---|---|---|---|
| | Mean | | Adjusted Titer | Log₁₀ |
| Sample | PFU | Dilution | (PFU) | Reduction |
| Stock Virus | 11.33 ± 1.43 | 1.0E+6 | 7.36 ± 0.05 | N/A |
| Control | | | | |
| T₀ | 25.00 ± 4.97 | 1.0E+3 | 6.70 ± 0.08 | 0.66 ± 0.09 |
| E-Beam | 0.00 | 1.0E+0 | <2.3 | >4.4 |
| Treated | | | | |
| E-Beam | 0.00 | 1.0E+0 | <2.3 | >4.4 |
| Untreated | | | | |
| Shipping | 25.33 ± 18.97 | 1.0E+4 | 6.70 ± 0.25 | 0.66 ± 0.25 |
| Control | | | | |

Viral titers were determined by multiplying the “Mean PFU” by the “Dilution” and dividing by a volume of 0.5 mL/well plated. The Logio Reduction values for the To and Shipping Control samples were calculated by subtracting the “Log10 Adjusted Titer” from that of the Stock Virus Control. The Logio Reduction values for the E-Beam Treated and E-Beam Untreated samples were calculated by subtracting the “Log10 Adjusted Titer” from that of the To sample.

The samples incubated with enveloped viruses (PrV and X-MuLV) and not irradiated had little to no detectible virus upon testing. By comparison, the “shipping control” samples that did not include test material and not irradiated had a majority of the virus remaining. For the samples incubated with the non-enveloped viruses (EMC and PPV) and not irradiated, virus was detected. These results suggest the spleen/lung ECM material provided antiviral activity for the enveloped viruses. While the material tested for this example comprised a 1:1 weight ratio of porcine spleen ECM material and porcine lung ECM material, it is believed that antiviral activity was not limited to one of these types of ECM materials, and that other compositions (including e.g., 100% spleen ECM material, 100% lung ECM material, as well as the other combinations and types of ECM materials disclosed herein) likewise are useful for treating viral infections.

It is intended that the specification and examples be considered as exemplary only, with a true scope and spirit of the present disclosure being indicated by the following claims.

What is claimed is:

1. A method of treating a subject, the method comprising:
administrating a composition to the subject, wherein the composition comprises at least one extracellular matrix (ECM) material, the at least one ECM material being derived from tissue chosen from spleen, lung, gall bladder, bone marrow, pancreas, liver, or a mixture thereof, the at least one ECM material retaining native bioactive components of the tissue;
wherein the subject is infected or suspected of being infected with a virus.

2. The method of claim 1, wherein the composition reduces a viral load of the subject.

3. The method of claim 1, wherein the subject has an infectious viral disease.

4. The method of claim 1, wherein the virus is an enveloped virus.

5. The method of claim 1, wherein the virus is a coronavirus or an adenovirus.

6. The method of claim 5, wherein the virus is severe acute respiratory syndrome-associated coronavirus (SARS-CoV), severe acute respiratory syndrome-associated coronavirus 2 (SARS-CoV-2), or Middle East respiratory syndrome coronavirus (MERS-CoV).

7. The method of claim 1, wherein the composition inactivates at least a portion of a virus population of the subject.

8. The method of claim 1, wherein the subject is a human or a non-human mammal.

9. The method of claim 1, wherein the composition comprises at least two different types of ECM materials.

10. The method of claim 1, wherein the composition comprises spleen ECM material and lung ECM material.

11. The method of claim 1, wherein the composition is in particulate form, gel form, or liquid form.

12. The method of claim 1, wherein the composition is administered via inhalation.

13. The method of claim 1, wherein the composition is administered intravenously.

14. A method of treating a subject, the method comprising:
administrating a composition to the subject, wherein the composition comprises at least two different extracellular matrix (ECM) materials, the ECM materials being derived from a tissue independently chosen from spleen tissue, lung tissue, gall bladder tissue, bone marrow tissue, pancreas tissue, or liver tissue;
wherein the subject is infected or suspected of being infected with a virus; and
wherein the virus is an enveloped virus.

15. The method of claim 14, wherein the virus is a coronavirus or an adenovirus.

16. The method of claim 14, wherein the subject has an infectious viral disease.

17. The method of claim 14, wherein the composition is administered via inhalation.

18. The method of claim 14, wherein the composition is administered intravenously.

19. A method of treating a subject, the method comprising:
administrating a composition to the subject, wherein the composition comprises at least one extracellular matrix (ECM) material, the at least one ECM material being derived from a tissue chosen from spleen tissue, lung tissue, gall bladder tissue, bone marrow tissue, pancreas tissue, or liver tissue;
wherein the subject is infected or suspected of being infected with a virus; and
wherein the subject is a human or a non-human mammal.

20. The method of claim 19, wherein the virus is a coronavirus or an adenovirus.
